(19) United States
(12) Patent Application Publication
(10) Pub. No.: US 2021/0163989 A1
(43) Pub. Date: Jun. 3, 2021

(54) TRANSDUCTION OF INNATE IMMUNOCOMPETENT CELLS USING AAV6

(71) Applicants: EMORY UNIVERSITY, Atlanta, GA (US); CHILDREN'S HEALTHCARE OF ATLANTA, INC., Atlanta, GA (US)

(72) Inventors: Harold Trent Spencer, Atlanta, GA (US); Christopher Doering, Atlanta, GA (US); George Aslanidi, Atlanta, GA (US)

(73) Assignees: EMORY UNIVERSITY, Atlanta, GA (US); CHILDREN'S HEALTHCARE OF ATLANTA, INC., Atlanta, GA (US)

(21) Appl. No.: 17/257,475

(22) PCT Filed: Jul. 5, 2019
(86) PCT No.: PCT/US2019/040742

§ 371 Date: Dec. 31, 2020

Related U.S. Application Data
(60) Provisional application No. 62/694,082, filed on Jul. 5, 2018.

(51) Int. Cl. C12N 15/86; A61K 35/17; A61K 45/06

(52) U.S. Cl. CPC C12N 15/86; A61K 35/17; C12N 2510/00; C12N 2750/14122; C12N 2750/14143; A61K 45/06

(57) ABSTRACT

Provided herein are recombinant AAV (rAAV) serotypes that are useful for targeting innate immune cells. In some embodiments, the rAAV are used to deliver genes encoding one or more receptors that can target the innate immune cells to diseased tissue of interest. In some aspects, the rAAV particle is a rAAV particle having a mutation in a surface-exposed amino acid, such as tyrosine, threonine, or serine, that enhances transduction of dendritic cells.

### CROSS-REFERENCE TO RELATED APPLICATIONS

This application claims the benefit of U.S. Provisional Application No. 62/694,082 filed Jul. 5, 2018. The entirety of this applications is hereby incorporated by reference for all purposes.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy and is hereby incorporated by reference into the specification. The name of the text file containing the Sequence Listing is 16101PCT_ST25.txt. The text file is 7 KB, was created on Jul. 5, 2019, and is being submitted electronically via EFSWeb.

### BACKGROUND

Although AAV6 vectors have been used successfully in a number of human cells (e.g., fibroblasts, dendritic, hematopoietic stem cells) and animal models (e.g., dog—muscle, cardiomyocytes; mouse—muscle, lungs), a number of steps in the life cycle of AAV, including intracellular trafficking and nuclear transport, continue to appear to limit the effectiveness of these vectors in gene therapy.

Pandya et al. report reprogramming immune response with capsid-optimized AAV6 vectors for immunotherapy of cancer. J Immunother. 2015, 38(7):292-8.

Wang et al. report homology-driven genome editing in human T cells by combining zinc-finger nuclease mRNA and AAV6 donor delivery. Nucleic Acids Res. 2016, 44(3):e30

Hale et al. report homology-directed recombination for enhanced engineering of chimeric antigen receptor T cells. Mol Ther Methods Clin Dev. 2017, 4:192-203. See also WO2017180989.

References cited herein are not an admission of prior art.

### SUMMARY

Aspects of the disclosure include a method of delivering a recombinant nucleic acid to innate immune cells by contacting the cells with a recombinant adeno-associated virus (rAAV). In some embodiments, the method comprises contacting the cells with a recombinant adeno-associated virus (rAAV) comprising the recombinant nucleic acid. In some embodiments, the rAAV VP3 capsid protein comprising a non-serine amino acid residue at a position corresponding to S663 of the wild-type AAV6 capsid protein as set forth in SEQ ID NO:1. In some embodiments, the non-serine amino acid residue at a position corresponding to S663 of the wild-type AAV6 capsid protein as set forth in SEQ ID NO:1 is valine.

In some embodiments, the nucleic acid is an expression construct that is flanked on each side by an inverted terminal repeat sequence. In some embodiments, the innate cells comprise natural killer cells and T cells. In some embodiments, the T cells are gamma delta (γδ) T cells.

In some embodiments, the rAAV are used to deliver genes encoding one or more receptors that can target the innate immune cells to diseased tissue of interest. In some embodiments, the recombinant nucleic acid encodes a chimeric antigen receptor (CAR).

In some embodiments, the recombinant nucleic acid encodes one or more genome editing proteins. In some embodiments, the genome editing protein can be nucleases, caspases, recombinases, and/or Cas9 proteins and variants and fusions thereof.

In some embodiments, the nucleic acid is a single-stranded or self-complementary rAAV nucleic acid vector. In some embodiments, the rAAV particle is an rAAV6 particle. In some embodiments, the recombinant nucleic acid is delivered in vivo or in vitro. In some embodiments, the disclosure comprises a composition comprising a rAAV particle described herein. In some embodiments, the composition further comprises a pharmaceutically acceptable carrier. In some embodiments, the composition further comprises an adjuvant.

Aspects of the disclosure include methods of providing an immunotherapy for treating cancer in a subject by delivering an rAAV particle or composition described in this application to a subject in an amount sufficient to produce an immunotherapeutic response in the subject. In some embodiments, the method further comprises delivering an adjuvant to the subject. In some embodiments, the method further comprises delivering a chemotherapeutic agent to the subject.

In some embodiments, the rAAV particle or composition is injected subcutaneously, intramuscularly, or intradermally. In some embodiments, subject was diagnosed as having cancer.

In some embodiments, the subject is at risk of developing cancer. In some embodiments, the cancer is selected from the group consisting of lymphomas, hemangiosarcomas, B-cell leukemia, mast cell tumors, osteosarcomas, melanomas, prostate cancer, thyroid cancer, liver cancer, pancreatic cancer, brain tumors, kidney cancer, ocular cancer, head or neck cancer, lung cancer, breast cancer, cervical cancer, gastrointestinal cancers, and urogenital cancers. In some embodiments, the subject is a mammal. In some embodiments, the subject is a human, a non-human primate, a companion animal, or a farm animal.

These and other aspects are described in more detail herein.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 shows a single-stranded AAV expression cassette: MND-GFP.

FIG. 2 shows a double-stranded AAV expression cassette: MND-GFP. (Astrakhan A, at al., Blood. 2012; Sather B D, et al., Sci Transl Med. 2015)

FIG. 3 shows PBMCs were obtained from two healthy donors and cultured in serum free media according published procedures in K. Sutton et al., Cytotherapy; 18:881-892; 2016, the entirety of which is incorporated herein. Flow cytometry was used to identify specific cellular lineages. CD3 was used as a marker for T cells, Pan γδ T cells were a marker for the innate T cell population γδ T cells, and CD56 is a marker for NK cells. For donor 1, it was shown that on day 10 of culture, there were approximately 52% γδ T cells, 37% T cells that were not γδ T cells and 9% NK cells. For donor 2, the values were approximately 48%, 32% and 19% respectively. The middle panel labeled “lenti” shows low transduction efficiency of γδ T cells (4.5% and 11.5% for donors 1 and 2 respectively). In contrast, wild type AAV6 (labeled as AAV6(wt)) showed high level transduction of 77% and 48% for donors 1 and 2 respectively. AAV6 S663V showed high level transduction as well, at 55% for donor 1.

FIG. 4 shows that AAV6 transduces innate NK cells much more efficiently than lentivirus. Transduction efficiency of primary human NK cells was shown by expanding PBMCs in serum free media (left panels) and adding recombinant lentivirus or AAV or an AAV6-S663V variant. The middle panels show that lentivirus transduction efficiency is low, at 12% and less. The efficiency of AAV6 transduction was approximately 72% for donor one and 39% for donor two. AAV6-S663V showed the greatest transduction efficiency at nearly 80% (right panel).

FIG. 5A shows a schematic representation of a bicistrionic transgene expressing GFP and an anti-CD5-CAR.

FIG. 5B shows transduction efficiency of the 5 innate NK cell line, NK-92 using lentiviral vector. The left panel C shows naïve NK-92 cells, the middle panel shows the low efficiency of lentiviral transduction (approximately 3%), and the right panel shows lentiviral transduced cells after flow sorting, which selects for modified cells.

FIG. 5C shows NK-92 cells were transduced with recombinant virus encoding the CD5-CAR and expression was verified by western blot analysis and flow cytometry. Transduction efficiency was less than 3%. GFP positive cells were flow sorted to greater than 90%, which was required to observe transgene expression by western blot analysis, which used an anti-CD3ζ antibody.

FIG. 5D shows transduction efficiency of the same cell line (top left panel C shows naïve NK-92 cells) using AAV6 (bottom left panel), and two variants an 6m3 (top right) and 663 (bottom right). Greater than 90% efficiency is achieved with each AAV6 vector.

### DETAILED DESCRIPTION

Provided herein are nucleic acids, recombinant adeno-associated virus (rAAV) particles, compositions, and methods for immunotherapy, for example for targeting innate immune cells.

Aspects of the present application are related to the surprising effectiveness of rAAV vectors (for example, but not limited to, capsid-modified AAV6 vectors) for inducing a protective immune response (e.g., in innate cells such as T cells and natural killer (NK) cells) in subjects, for example subjects having cancer. In some embodiments, the substitution of specific surface exposed specific tyrosine (Y), serine (S), and threonine (T) residues on AAV6 capsids significantly increases the transduction efficiency of these vectors. In other embodiments, phenylalanine (F) and valine (V) were used for substitution due to their opposite charge. As described herein, rAAV vectors can be used to deliver cancer-specific antigens to a subject (e.g., to antigen presenting cells in the subject) to produce a protective response in the subject. In some aspects, rAAV vectors described herein provide a good balance of immunogenicity and high transduction efficiency (e.g., using capsid-optimized vectors such as AAV6-Y705-731F+T492V, (Sayroo R, et al., Gene Therapy, 2015; Rosario A M, et al, Mol Ther Methods Clin Dev., 2016) and AAV6-S663V (Pandya J, et al., Immunology and Cell Biol., 2014) for delivering cancer associated antigens to a subject in a therapeutically effective amount.

In some aspects, the application relates to recombinant AAV (rAAV) serotypes that are useful for targeting innate immune cells. In some embodiments, the rAAV are used to deliver genes encoding one or more receptors that can target the innate immune cells to diseased tissue of interest. In some embodiments, the tissue of interest includes tumor tissue. In some embodiments, the rAAV kill or prevent the tissue of interest from proliferating. In some embodiments, the gene being delivered is a receptor for a diseases tissue of interest. In some embodiments, the innate cells are targeted to the diseased tissue to prevent further proliferation and/or killing of the target tissue.

In some embodiments, the rAAV are rAAV6. In some embodiments, the rAAV6 have one or more capsid mutations. In some embodiments, the innate cells are T cells or NK cells. In some embodiments, the receptors are innate cells.

In some embodiment, innate cells are isolated and/or separated using multicolor flow cytometry.

In some embodiment, the modified cells are delivered in vivo or in vitro. In some embodiments, the modified cells are reintroduced into patients.

In some embodiments, innate cells are expanded from blood, bone marrow or cord blood. Methods of expansion are standard for the field, and may use specific cytokines for various innate cell lineages. For example, in some embodiments, IL-2 is useful for the expansion of γδ T cells. In some embodiments, IL-15 is useful for the expansion of NK cells. In some embodiments, a priming peptide is useful for the expansion of Natural Killer T (NKT). In some embodiments, additives such as bisphosphates may be added. For example, the expansion of y6 T cells cultures may use zoledronic acid.

In some embodiments, γδ cells are isolated using TCRγ/δ+ T Cell Isolation Kits. In some embodiments, a cell sample is prepared, non-T cells are magnetically labeled, magnetic separation is sued to deplete non-T cells from the sample (e.g., using an LD column or an autoMACS™ separator, γδ cells (e.g., TCR γδ + T cells) magnetic labeling of the γδ cells, and magnetic separation for positive selection of the γδ cells (e.g., positive selection with MS columns or positive selection with the autoMACS™ separator).

In some embodiments, the modified cells are delivered in vivo or in vitro. In some embodiments, the modified cells are reintroduced into patients. In some embodiments, the modified cells are used for systemic treatments (e.g., such as for disseminated cancers) thr infusion of cells may be intravenous. Alternatively, in some embodiments, AAV6 modified cells may be administered directly to the tumor, for example, for solid tumors. Alternatively, in some embodiments, AAV6 modified cells may be used in vitro. For example, mixing AAV6 modified cells in vitro with other therapeutic cells prior to infusion can, for example, be used to purge the therapeutic cells of unwanted cells.

In some embodiments, treatment is combined with other therapies. In some embodiments, treatment is combined with chemotherapy, checkpoint inhibitors, monoclonal antibody treatment, hormonal treatment, radiation, surgery, or other treatments.

### T Cells

Aspects of the application relate to targeting innate immune cells including innate T cells or T lymphocytes. T cells or T lymphocytes play a central role in cell-mediated immunity. T cells are distinguished from other lymphocytes, (e.g., B cells and natural killer cells) by the presence of a T-cell receptor on the cell surface. Subsets of T cells, including αβ T cells and γδ T cells, have a distinct function. The majority of human T cells rearrange their alpha and beta chains on the cell receptor, termed alpha beta T cells (αβ T cells), and are part of the adaptive immune system. Specialized gamma delta T cells, a minority of T cells in the human body, have invariant T cell receptors with limited diversity that can effectively present antigens to other T cells and are considered to be part of the innate immune system.

Gamma delta T cells (γδ T cells), represent a small subset of innate T cells that possess a distinct T-cell receptor (TCR) on their surface. Gamma delta T cells (γδ T cells) are T cells that express a unique T-cell receptor (TCR) composed of one γ-chain and one δ-chain. Gamma delta T cells are found in the gut mucosa, skin, lungs and uterus, and are involved in the initiation and propagation of immune responses.

CD3 is expressed on all T cells as it is associated with the T cell receptor (TCR). The majority of TCR are made up of alpha beta chains (alpha beta T-cells). Alpha beta T-cells and gamma delta T cells are believed to be derived from a common CD4⁻CD8⁻ double-negative precursor thymocytes. Mature gamma delta T cells are CD4⁻CD8⁻ double-negative. In contrast, alpha beta T-cells typically become double-positive intermediates (CD4⁺CD8⁺) which mature into single-positive (CD4⁺CD8⁻) T helper cells or (CD4⁻CD8⁺) cytotoxic T cells. Memory cells may be either CD4⁺ or CD8⁺. Memory T cells typically express the cell surface protein CD45RO. T cells may be isolated and separated from a human sample (blood or PBMCs) based on the expression of alpha beta T cell receptor (TCR), gamma delta T cell receptor, CD2, CD3, CD4, CD8, CD4 and CD8, NK1.1, CD4 and CD25 and other combinations based on positive or negative selection. TCRγ/δ⁺ T cells are typically TCRα/β−, CD2⁺, CD3⁺, and CD5⁺ See also Salot et al., Large scale expansion of Vgamma9Vdelta2 T lymphocytes from human peripheral blood mononuclear cells after a positive selection using MACS “TCR gamma/delta⁺ T cell isolation kit,” J Immunol Methods, 2009, 347(1-2):12-8.

### Chimeric Antigen Receptor (CAR) Modified T Cells (CARTs)

In some aspects, rAAV are used to deliver one or more genes that encode chimeric antigen receptor(s) to innate immune cells (e.g., to produce chimeric antigen receptor modified T cells). Chimeric antigen receptor (CAR) modified T cells (CARTs) have great potential in selectively targeting specific cell types, and utilizing the immune system surveillance capacity and potent self-expanding cytotoxic mechanisms against tumor cells with exquisite specificity. This technology provides a method to target neoplastic cells with the specificity of monoclonal antibody variable region fragments, and to affect cell death with the cytotoxicity of effector T cell function. For example, the genes delivered are the single genes that have an antigen receptor fused to a transmembrane domain. The antigen receptor can be a scFv or any other monoclonal antibody domain.

CARs are bio-engineered receptors that confer targeting specificity for immune cells by grafting the single chain variable fragments (scFv) of monoclonal antibody domains onto to the CD3-zeta transmembrane and endodomain from endogenous T cell receptors (TCRs). This chimeric cell surface protein results in the transmission of a zeta signal in response to recognition by the scFv of the target antigen. Activation of T cells in turn triggers immunological cascades resulting in local cell death. Recombinant genes in the rAAV system can encode a CAR, which can be an antigen receptor fused to a transmembrane protein.

CART therapy has been successfully applied for the treatment of several different tumor types, which had been refractory to other forms of treatment. A remarkable success of CART therapy has been demonstrated in the treatment of chronic lymphocyte leukemia. Accordingly, in some embodiments, rAAV described herein, can be used to treat refractory forms of cancer including leukemia (e.g., B-cell leukemia or T-cell leukemia). In some embodiments, rAAV described herein, can be used to treat refractory forms of cancer including neuroblastoma.

A key to CART therapy is the availability of cell surface antigens that can be targeted in a cell specific manner. There are several cell surface antigens that are selectively expressed. In some embodiments, the present invention relates, in part, to the use of T cells genetically modified to stably express a desired CAR, (e.g., containing a IL-15Rα cytoplasmic domain). T cells expressing a CAR are referred to herein as CAR T cells, CARTs, or CAR modified T cells. Preferably, the cell can be genetically modified to stably express an antibody binding domain on its surface, conferring novel antigen specificity that is MHC independent. In some instances, the T cell is genetically modified to stably express a CAR that combines an antigen recognition domain of a specific antibody with a transmembrane domain and a cytoplasmic domain into a single chimeric protein. In some embodiments, two CAR proteins dimerize (e.g., form homo- or heterodimers) in vivo.

In certain embodiments, this disclosure relates to rAAV disclosed herein comprising a nucleic acid that encodes a chimeric polypeptide comprising a targeting sequence, a transmembrane domain, a T cell costimulatory molecule domain, and a signal-transduction component of a T-cell antigen receptor domain such as CD3zeta (CD3Z).

In certain embodiments, the costimulatory molecule is selected from CD28, CD80, CD86 or fragment or variant. In certain embodiments, the signal-transduction component of the T-cell antigen receptor comprises an immunoreceptor tyrosine-based activation motif. In certain embodiments, the rAAV further comprises an interleukin sequence such as IL-2 or fragment or variant. In certain embodiments, the recombinant vector further comprises CD8 or fragment or variant.

In certain embodiments, the rAAV further comprises a nucleic acid encoding an enzyme that confers resistance to cellular damage in the presence of a chemotherapy agent. In certain embodiments, the rAAV further comprises a nucleic acid encoding methylguanine methyltransferase (MGMT), dihydrofolate reductase (DHFR), cytidine deaminase (CD), and multidrug resistant protein (MDR-1) or variants thereof.

In certain embodiments, the rAAV further comprises a nucleic acid encoding the targeting sequence that specifically binds to a tumor associated antigen such as CD5, CD19, CD20, CD30, CD33, CD47, CD52, CD152(CTLA-4), CD274(PD-L1), CD340(ErbB-2), GD2, TPBG, CA-125, CEA, MAGEA1, MAGEA3, MART 1, GP100, MUC1, WT1, TAG-72, HPVE6, HPVE7, BING-4, SAP-1, immature laminin receptor, vascular endothelial growth factor (VEGF-A) or epidermal growth factor receptor (ErbB-1).

In some embodiments, aspects of the disclosure relate to the delivery of proteins, such as genome editing proteins, to target cells. The present invention includes complexes, compositions, and preparations, for the delivery of one or more functional effector proteins, e.g., nucleases, recombinases, and Cas9 proteins (including variants and fusions thereof, e.g., Cas9 nickases and Cas9 fusions to deaminases, gene editing enzymes, transcriptional repressors and activators, epigenetic modifiers, etc.), to a cell. In some embodiments, the genome editing proteins include Zinc Finger Nucleases (ZFNs), TALENs, CRISPR/Cas proteins, and/or meganucleases. In some embodiments, the cell is an innate cell. In some embodiments, the biological effect exerts a therapeutic benefit to a subject in which the cell is found. The complexes, compositions, preparations, systems, kits, and related methods for delivery of functional effector proteins are useful for introducing an effector protein into a cell, e.g., in the context of manipulating the cell for a research or therapeutic purpose. delivery of site-specific proteins, such as TALENs or Cas9 proteins (or variants or fusions thereof) using the compositions, preparations, systems, kits, and related methods provided herein allows for the targeted manipulation/modification of the genome of a host cell in vitro or in vivo.

Methods described by the disclosure may be used to deliver proteins into cells for a variety of purposes, such as delivery of therapeutic proteins, or genome editing. As used herein, “genome editing” refers to the adding, disrupting or changing the sequence of specific genes by insertion, removal or mutation of DNA from a genome using artificially engineered proteins and related molecules. For example, genome editing proteins, such as TALENS, may be delivered to a cell using a method described herein. The TALEN can introduce double stranded breaks at a target locus in the host cell genome, resulting in altered gene function and/or expression. TALENS can also promote DNA repair (e.g., non-homologous end joining or homology-directed repair), which is useful for rescue construct-mediated stable integration of foreign genetic material into the genome of a host cell. Rescue constructs, comprising a polynucleotide encoding a desired insertion or mutation, can be delivered before, after, or simultaneously to a genome editing protein in order to introduce a mutation or other alteration at the target locus. A rescue construct can be single-stranded polynucleotide or a double-stranded polynucleotide. In some embodiments, a rescue construct is a single-stranded oligonucleotide DNA (ssODN). In some embodiments, a rescue construct is a plasmid, viral vector, or interfering RNA (dsRNA, siRNA, shRNA, miRNA, AmiRNA, etc.). The one or more isolated cells can be transfected with a rescue construct before, after or simultaneous to contact with the bacterium. In some embodiments, the rescue construct is delivered separately from the bacterium. In some embodiments, the rescue construct is expressed by the bacterium.

In some embodiments, an rAAV composition described herein (e.g., a mutant AAV6) is used to deliver a gene that expresses a suitable target for immunotherapy, for example an antigen or epitope that is characteristic of a cancer being treated. In some embodiments, the antigen or epitope is a marker that is unique to the cancer of interest. In some embodiments, the antigen or epitope is a marker that is over-expressed in the cancer of interest. Non-limiting examples of targets for immunotherapy include prostatic acid phosphatase for prostate cancer and FMS-like tyrosine kinase 3 ligand for B cell lymphoma. However, other proteins or peptides can be delivered for immunotherapy as described herein.

In some embodiments, rAAV vectors (e.g., capsid-modified AAV vectors) can transduce different subsets of innate cells or combinations thereof in a subject after direct administration to the subject (e.g., intradermally, subcutaneously, intramuscularly, or via any other suitable route as described in more detail herein).

In some embodiments, compositions described herein can be administered to subjects having cancer (e.g., diagnosed as having cancer) to treat or help treat the cancer (for example, alone or in conjunction with one or more additional anti-cancer therapies). In some embodiments, composition described herein can be administered to prevent or help prevent the spread of a cancer or the further growth of a tumor. In some embodiments, one or more compositions described herein are administered to a subject as a vaccine for preventing formation of solid tumors and/or metastasis. In some embodiments, one or more compositions described herein can be administered to a subject post-surgery (or after other treatment), for example to reduce the risk or prevent recurrence of a cancer.

In some embodiments, compositions described herein can be administered as a vaccine to a subject (e.g., a subject at risk of cancer, for example due to one or more genetic risk factors, or due to exposure to one or more carcinogens and/or radiation) to reduce the risk or prevent the occurrence of a cancer.

Accordingly, in some embodiments aspects of the disclosure can be used for immunotherapy to treat one or more cancers. In some embodiments, rAAV compositions described herein may need to be administered to a subject more than once (for example to support an initial treatment by providing an immunotherapeutic boost at one or more later dates). In some embodiments, a different AAV serotype (or different capsid variants of an rAAV) are used for the different administrations.

In some embodiments, rAAV variants with increased efficiency of transducing nucleic acids into the nucleus of a target cell (e.g., as a result of reduced proteasomal degradation relative to wild-type AAV capsids) can be used.

In some embodiments, rAAV vectors described herein can promote mild inflammation that can promote maturation of target dendritic cells (or other target cells).

In some embodiments, one or more compositions described herein are administered along with an adjuvant, a chemotherapeutic drug, other cancer treatment, or a combination of two or more thereof as described in more detail herein.

Recombinant Adeno-Associated Virus (rAAV) Particles and Nucleic Acids

The term “a nucleic acid sequence encoding” a specified polypeptide refers to a nucleic acid sequence comprising the coding region of a gene or in other words the nucleic acid sequence which encodes a gene product. The coding region may be present in a cDNA, genomic DNA or RNA form. When present in a DNA form, the oligonucleotide, polynucleotide, or nucleic acid may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present disclosure may contain endogenous enhancers/promoters, splice junctions, intervening sequences, polyadenylation signals, etc. or a combination of both endogenous and exogenous control elements.

The term “recombinant” when made in reference to a nucleic acid molecule refers to a nucleic acid molecule which is comprised of segments of nucleic acid joined together by means of molecular biological techniques. The term “recombinant” when made in reference to a protein or a polypeptide refers to a protein molecule which is expressed using a recombinant nucleic acid molecule. The term recombinant nucleic acid is distinguished from the natural recombinants that result from crossing-over between homologous chromosomes. Recombinant nucleic acids as used herein are an unnatural union of nucleic acids from nonhomologous sources, i.e., heterologous, usually from different organisms.

The terms “vector” or “expression vector” refer to a recombinant nucleic acid containing a desired coding sequence and appropriate nucleic acid sequences necessary for the expression of the operably linked coding sequence in a particular host organism or expression system, e.g., cellular or cell-free. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

Protein “expression systems” refer to in vivo and in vitro (cell free) systems. Systems for recombinant protein expression typically utilize non-zygotic cells transfecting with a DNA expression vector that contains the template. The cells are cultured under conditions such that they translate the desired protein. Expressed proteins are extracted for subsequent purification. In vivo protein expression systems using prokaryotic and eukaryotic cells are well known. Also, some proteins are recovered using denaturants and protein-refolding procedures. In vitro (cell-free) protein expression systems typically use translation-compatible extracts of whole cells or compositions that contain components sufficient for transcription, translation and optionally post-translational modifications such as RNA polymerase, regulatory protein factors, transcription factors, ribosomes, tRNA cofactors, amino acids and nucleotides. In the presence of an expression vectors, these extracts and components can synthesize proteins of interest. Cell-free systems typically do not contain proteases and enable labeling of the protein with modified amino acids. Some cell free systems incorporated encoded components for translation into the expression vector. See, e.g., Shimizu et al., Cell-free translation reconstituted with purified components, 2001, Nat. Biotechnol., 19, 751-755 and Asahara & Chong, Nucleic Acids Research, 2010, 38(13): e141, both hereby incorporated by reference in their entirety.

A “selectable marker” is a nucleic acid introduced into a recombinant vector that encodes a polypeptide that confers a trait suitable for artificial selection or identification (report gene), e.g., beta-lactamase confers antibiotic resistance, which allows an organism expressing beta-lactamase to survive in the presence antibiotic in a growth medium. Another example is thymidine kinase, which makes the host sensitive to ganciclovir selection. It may be a screenable marker that allows one to distinguish between wanted and unwanted cells based on the presence or absence of an expected color. For example, the lac-z-gene produces a beta-galactosidase enzyme which confers a blue color in the presence of X-gal (5-bromo-4-chloro-3-indolyl-β-D-galactoside). If recombinant insertion inactivates the lac-z-gene, then the resulting colonies are colorless. There may be one or more selectable markers, e.g., an enzyme that can complement to the inability of an expression organism to synthesize a particular compound required for its growth (auxotrophic) and one able to convert a compound to another that is toxic for growth. URA3, an orotidine-5′ phosphate decarboxylase, is necessary for uracil biosynthesis and can complement ura3 mutants that are auxotrophic for uracil. URA3 also converts 5-fluoroorotic acid into the toxic compound 5-fluorouracil. Additional contemplated selectable markers include any genes that impart antibacterial resistance or express a fluorescent protein. Examples include, but are not limited to, the following genes: amp^{r}, cam^{r}, tet^{r}, blasticidin^{r}, neo^{r}, hyg^{r}, abx^{r}, neomycin phosphotransferase type II gene (nptII), p-glucuronidase (gus), green fluorescent protein (gfp), egfp, yfp, mCherry, p-galactosidase (lacZ), lacZa, lacZAM15, chloramphenicol acetyltransferase (cat), alkaline phosphatase (phoA), bacterial luciferase (luxAB), bialaphos resistance gene (bar), phosphomannose isomerase (pmi), xylose isomerase (xylA), arabitol dehydrogenase (atlD), UDP-glucose:galactose-1-phosphate uridyltransferasel (galT), feedback-insensitive α subunit of anthranilate synthase (OASA1D), 2-deoxyglucose (2-DOGR), benzyladenine-N-3-glucuronide, E. coli threonine deaminase, glutamate 1-semialdehyde aminotransferase (GSA-AT), D-amino acidoxidase (DAAO), salt-tolerance gene (rstB), ferredoxin-like protein (pflp), trehalose-6-P synthase gene (AtTPS1), lysine racemase (lyr), dihydrodipicolinate synthase (dapA), tryptophan synthase beta 1 (AtTSB1), dehalogenase (dhlA), mannose-6-phosphate reductase gene (M6PR), hygromycin phosphotransferase (HPT), and D-serine ammonialyase (dsdA).

Aspects of the disclosure relate to recombinant AAV (rAAV) particles and nucleic acids. In some embodiments, a nucleic acid is provided, the nucleic acid comprising an expression construct containing a truncated promoter operably linked to a coding sequence of a gene of interest. In some embodiments, a promoter is a natural or heterologous promoter. In some embodiments, a promoter can be a truncated natural promoter. In some embodiments, a promoter can include an enhancer and/or basal promoter elements from a natural promoter. In some embodiments, a promoter can be or include elements from a CMV, a chicken beta actin, a desmin, or any other suitable promoter or combination thereof. In some embodiments, a promoter can be an engineered promoter. In some embodiments, a promoter is transcriptionally active in dendritic cells. In some embodiments, a promoter is less than 1.6 kb in length, less than 1.5 kb in length, less than 1.4 kb in length, less than 1.3 kb in length, less than 1.2 kb in length, less than 1.1 kb in length, less than 1 kb in length, or less than 900 kb 900 bp in length.

In some embodiments, an expression construct including a promoter and a gene of interest is flanked on each side by an inverted terminal repeat sequence.

The coding sequence of a gene of interest may be any coding sequence of any gene that is appropriate for use in immunotherapy. In some embodiments, the gene of interest is a gene that encodes a cancer associated antigen, for example a marker characteristic of a particular cancer. In some embodiments, the marker is unique to cancer cells (e.g., a mutant protein). In some embodiments, the marker is overexpressed in cancer cells relative to healthy cells. In some embodiments, the marker is a cell surface marker. Non-limiting example of genes of interest for treating prostate cancer as described herein include Prostatic Acid Phosphatase (PAP), Prostate specific antigen (PSA), and/or Prostate-specific membrane antigen (PSMA). Non-limiting example of genes of interest for treating breast cancer as described herein include Cancer Antigen 15-3 (CA-15.3), and/or Epidermal growth factor receptor 2 (Her2/neu). Non-limiting example of genes of interest for treating B cell lymphoma as described herein include FMS-like tyrosine kinase 3 ligand (FLT3). Non-limiting example of genes of interest for treating liver cancer include Alpha-fetoprotein (AFP), Hepatocyte growth factor receptor (HGFR, c-Met), and/or Glypican 3 (GLP3). Other non-limiting example of genes of interest for treating cancer include Carcinoembryonic antigen (CEA), and/or Telomerase (TERT). Other cancer markers can be used.

In some embodiments, the expression construct comprises one or more regions comprising a sequence that facilitates expression of the coding sequence of the gene of interest, e.g., expression control sequences operably linked to the coding sequence. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

In some embodiments, the nucleic acid is a plasmid (e.g., a circular nucleic acid comprising one or more of an origin of replication, a selectable marker, and a reporter gene). In some embodiments, a nucleic acid described herein, such as a plasmid, may also contain marker or reporter genes, e.g., LacZ or a fluorescent protein, and an origin of replication. In some embodiments, the plasmid is transfected into a producer cell that produces AAV particles containing the expression construct.

In some embodiments, the nucleic acid is a nucleic acid vector such as a recombinant adeno-associated virus (rAAV) vector. Exemplary rAAV nucleic acid vectors useful according to the disclosure include single-stranded (ss) or self-complementary (sc) AAV nucleic acid vectors.

In some embodiments, a recombinant rAAV particle comprises a nucleic acid vector, such as a single-stranded (ss) or self-complementary (sc) AAV nucleic acid vector. In some embodiments, the nucleic acid vector contains an expression construct as described herein and one or more regions comprising inverted terminal repeat (ITR) sequences (e.g., wild-type ITR sequences or engineered ITR sequences) flanking the expression construct. In some embodiments, the nucleic acid is encapsidated by a viral capsid.

Accordingly, in some embodiments, a rAAV particle comprises a viral capsid and a nucleic acid vector as described herein, which is encapsidated by the viral capsid. In some embodiments, the viral capsid comprises 60 capsid protein subunits comprising VP1, VP2 and VP3. In some embodiments, the VP1, VP2, and VP3 subunits are present in the capsid at a ratio of approximately 1:1:10, respectively.

The ITR sequences of a nucleic acid or nucleic acid vector described herein can be derived from any AAV serotype (e.g., 1, 2, 3, 4, 5, 6, 7, 8, 9, or 10) or can be derived from more than one serotype. In some embodiments of the nucleic acid or nucleic acid vector provided herein, the ITR sequences are derived from AAV2. In some embodiments of the nucleic acid or nucleic acid vector provided herein, the ITR sequences are derived from AAV6. ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Calif.; and Addgene, Cambridge, Mass.; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93(24):14082-7; and Curtis A. Machida. Methods in Molecular Medicine™. Viral Vectors for Gene Therapy Methods and Protocols. Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some embodiments, the expression construct is no more than 7 kilobases, no more than 6 kilobases, no more than 5 kilobases, no more than 4 kilobases, or no more than 3 kilobases in size. In some embodiments, the expression construct is between 4 and 7 kilobases in size.

The rAAV particle may be of any AAV serotype (e.g., 1, 2, 3, 4, 5, 6, 7, 8, 9, or 10), including any derivative (including non-naturally occurring variants of a serotype) or pseudotype.

In some embodiments, the rAAV particle is an rAAV6 particle. In some embodiments, the rAAV particle is an rAAV2 particle. Non-limiting examples of derivatives and pseudotypes include AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y→F), AAV8 (Y733F), AAV2.15, AAV2.4, AAVM41, and AAVr3.45. Such AAV serotypes and derivatives/pseudotypes, and methods of producing such derivatives/pseudotypes are known in the art (see, e.g., Mol Ther. 2012 April; 20(4):699-708. The AAV vector toolkit: poised at the clinical crossroads. Asokan Al, Schaffer D V, Samulski R J.).

In some embodiments, the rAAV particle comprises a capsid that includes modified capsid proteins (e.g., capsid proteins comprising a modified VP3 region). Methods of producing modified capsid proteins are known in the art (see, e.g., U.S. Patent Publication Number US20130310443, which is incorporated herein by reference in its entirety). In some embodiments, the rAAV particle comprises a modified capsid protein comprising a (i.e., at least one) non-native amino acid substitution at a position that corresponds to a surface-exposed amino acid in a wild-type capsid protein (e.g., wild-type AAV6 capsid protein, such as SEQ ID NO: 1, or other wild-type AAV capsid protein). In some embodiments, the rAAV particle comprises a modified capsid protein comprising a non-tyrosine amino acid (e.g., a phenylalanine) at a position that corresponds to a surface-exposed tyrosine amino acid in a wild-type capsid protein, a non-threonine amino acid (e.g., a valine) at a position that corresponds to a surface-exposed threonine amino acid in the wild-type capsid protein, a non-lysine amino acid (e.g., a glutamic acid) at a position that corresponds to a surface-exposed lysine amino acid in the wild-type capsid protein, a non-serine amino acid (e.g., a valine) at a position that corresponds to a surface-exposed serine amino acid in the wild-type capsid protein, or a combination thereof.

Exemplary surface-exposed amino acids include positions that correspond to S663, S551, Y705, Y731, and T492 of the wild-type AAV6 capsid protein. In some embodiments, a rAAV particle (e.g., a rAAV6 or other rAAV serotype particle) comprises a capsid that includes modified capsid proteins having one or more, for example two or more (e.g., 2, 3, 4, 5, or more) amino acid substitutions. Non-limiting examples of modified AAV6 capsid proteins include S663V+T492V, S663-551V, Y705-731F+T492V.

An exemplary, non-limiting wild-type AAV6 capsid protein sequence is provided below (SEQ ID NO: 1).

| | |
|---|---|
| 1 | MAADGYLPDWLEDNLSEGIREWWDLKPGAPKPKANQQKQDDGRGLVLPGY |
| 51 | KYLGPFNGLD KGEPVNAADAAALEHDKAYDQQLKAGDNPYLRYNHADAEF |
| 101 | QERLQEDTSF GGNLGRAVFQ AKKRVLEPFG LVEEGAKTAPGKKRPVEQSP |
| 151 | QEPDSSSGIG KTGQQPAKKR LNFGQTGDSE SVPDPQPLGEPPATPAAVGP |
| 201 | TTMASGGGAPMADNNEGADGVGNASGNWHCDSTWLGDRVITTSTRTWALP |
| 251 | TYNNHLYKQI SSASTGASND NHYFGYSTPW GYFDFNRFHCHFSPRDWQRL |
| 301 | INNNWGFRPK RLNFKLFNIQ VKEVTTNDGV TTIANNLTSTVQVFSDSEYQ |
| 351 | LPYVLGSAHQ GCLPPFPADV FMIPQYGYLT LNNGSQAVGRSSFYCLEYFP |
| 401 | SQMLRTGNNF TFSYTFEDVP FHSSYAHSQS LDRLMNPLIDQYLYFLNRTQ |
| 451 | NQSGSAQNKD LLFSRGSPAG MSVQPKNWLP GPCYRQQRVSKTKTDNNNSN |
| 501 | FTWTGASKYN LNGRESIINP GTAMASHKDD KDKFFPMSGV MIFGKESAGA |
| 551 | SNTALDNVMI TDEEEIKATN PVATERFGTV AVNLQSSSTD PATGDVHVMG |
| 601 | ALPGMVWQDR DVYLQGPIWA KIPHTDGHFH PSPLMGGFGL KHPPPQILIK |
| 651 | NTPVPANPPA EFSATKFASF ITQYSTGQVS VEIEWELQKE NSKRWNPEVQ |
| 701 | YTSNYAKSAN VDFTVDNNGL YTEPRPIGTR YLTRPL |

In certain embodiments, this disclosure relates to a non-naturally nucleic acid encoding an AAV6 capsid protein sequence as provided in SEQ ID NO: 1 with one or more of the following mutations: T251V, D328I, S455V, G470V, S472V, V489I, T492V, D495V, D495I, S499V, H527T, W503I, D530V, K531E, V540I, S551V, D590V, K666E, K567E, I648V, S663V, A664V, T665V, S669V, E689D, Y705F, A706I, A709V, A709I, V711I, V715I, T722V, Y731F, or combinations thereof. In certain embodiments, this disclosure relates to vectors comprising a nucleic acid encoding an AAV6 capsid protein in operable combination with a heterologous promoter. In certain embodiments, this disclosure relates to an expression system comprising a vector disclosed herein.

Methods of producing rAAV particles and nucleic acid vectors are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, the nucleic acid vector (e.g., as a plasmid) may be combined with one or more helper plasmids, e.g., that contain a rep gene (e.g., encoding Rep78, Rep68, Rep52 and Rep40) and a cap gene (encoding VP1, VP2, and VP3), and transfected into a producer cell line such that the rAAV particle can be packaged and subsequently purified.

In some embodiments, the one or more helper plasmids includes a first helper plasmid comprising a rep gene and a cap gene and a second helper plasmid comprising other genes that assist in AAV production, such as a E1a gene, a E1b gene, a E4 gene, a E2a gene, and a VA gene. In some embodiments, the rep gene is a rep gene derived from AAV2 and the cap gene is derived from AAV5. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Calif.; and Addgene, Cambridge, Mass.; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. Alternatively, in another example, Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known in the art or described herein, e.g., by iodixanol step gradient, CsCl gradient, chromatography, or polyethylene glycol (PEG) precipitation.

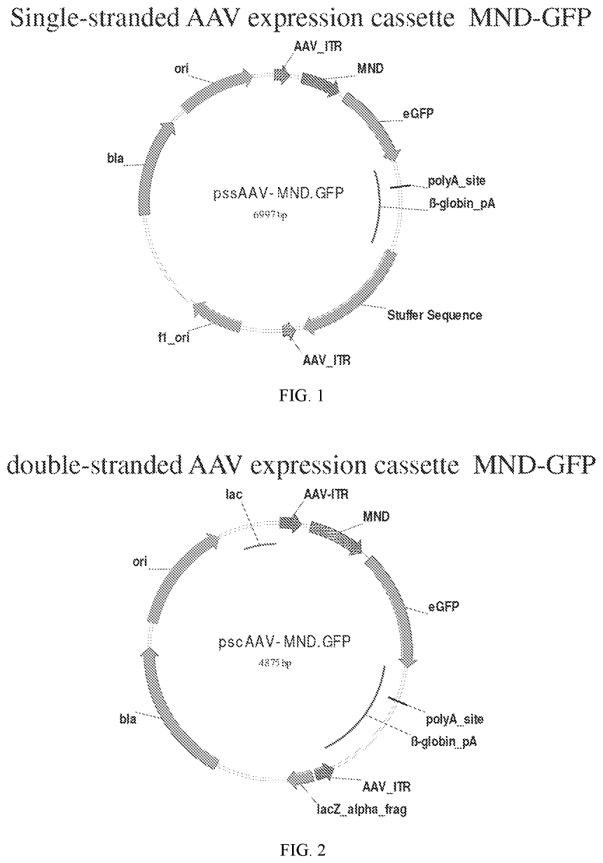

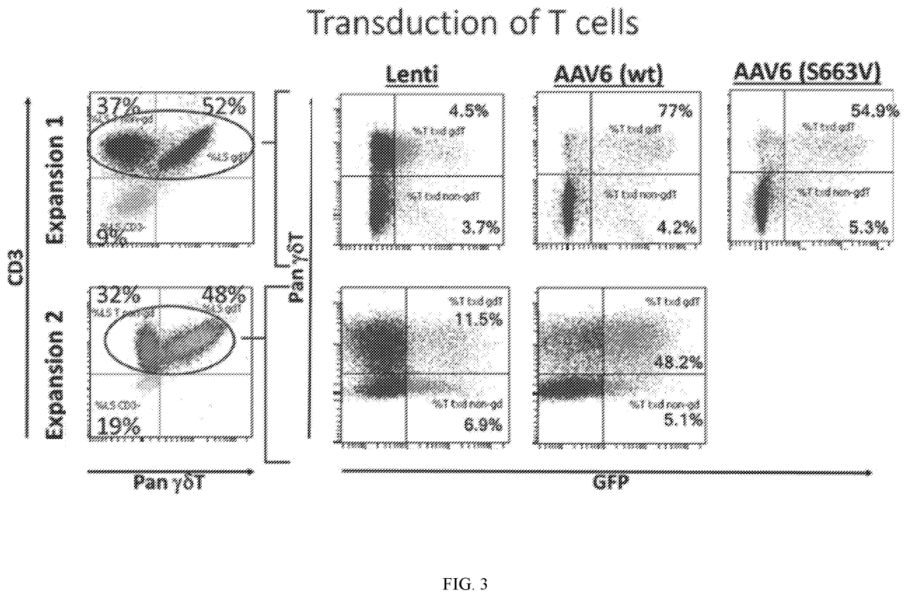

In some embodiments, MND promoter AAV expression cassettes are used. In some embodiments, MND-GFP AAV expression cassettes are used. Non-limiting examples of MND promoter expression cassettes can be found in Astrakhan A, et al., Blood. 2012 and Sather B D, et al., Sci Transl Med. 2015, the entire contents of each of which are incorporated herein by reference. Non-limiting examples of expression cassettes are shown in FIGS. 1 and 2. In some embodiments, one or more other genes of interest can be substituted for one or more genes illustrated in FIGS. 1 and 2. In some embodiments, one 5 or more genes of interest (e.g., a CAR gene) is included on a recombinant nucleic acid (e.g., in an expression cassette and/or in a rAAV genome) without one or more other exogenous genes (e.g., without a reporter gene such as a GFP gene).

The disclosure also contemplates host cells that comprise at least one of the disclosed rAAV particles, expression constructs, or nucleic acid vectors. Such host cells include mammalian host cells, with human host cells being preferred, and may be either isolated, in cell or tissue culture. In the case of genetically modified animal models (e.g., a mouse), the transformed host cells may be comprised within the body of a non-human animal itself.

### Compositions

Aspects of the disclosure relate to compositions comprising rAAV particles or nucleic acids described herein. In some embodiments, rAAV particles described herein are added to a composition, e.g., a pharmaceutical composition.

In some embodiments, the composition comprises a pharmaceutically acceptable carrier. The term “carrier” refers to a diluent, adjuvant, excipient, or vehicle with which the rAAV particle is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of pharmaceutically acceptable carriers include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, polyacrylic acids, lubricating agents (such as talc, magnesium stearate, and mineral oil), wetting agents, emulsifying agents, suspending agents, preserving agents (such as methyl-, ethyl-, and propyl-hydroxy-benzoates), and pH adjusting agents (such as inorganic and organic acids and bases). Other examples of carriers include phosphate buffered saline, HEPES-buffered saline, and water for injection, any of which may be optionally combined with one or more of calcium chloride dihydrate, disodium phosphate anhydrous, magnesium chloride hexahydrate, potassium chloride, potassium dihydrogen phosphate, sodium chloride, or sucrose. Other examples of carriers that might be used include saline (e.g., sterilized, pyrogen-free saline), saline buffers (e.g., citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of rAAV particles to human subjects. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Methods for making such compositions are well known and can be found in, for example, Remington: The Science and Practice of Pharmacy, 22nd edition, Pharmaceutical Press, 2012.

Typically, such compositions may contain at least about 0.1% of the therapeutic agent (e.g., rAAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) (e.g., rAAV particle) in each therapeutically-useful composition may be prepared ins such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, a composition described herein may be administered to a subject in need thereof, such as a subject having a cancer. In some embodiments, a method described herein may comprise administering a composition comprising rAAV particles as described herein to a subject in need thereof. In some embodiments, the subject is a human subject. In some embodiments, the subject has or is suspected of having a disease that may be treated with immunotherapy, such as cancer. In some embodiments, the subject has been diagnosed with cancer. In some embodiments, the subject is known to be at risk of having or developing cancer.

In some embodiments, a composition also comprises one or more adjuvants. Non-limiting examples of adjuvants include one or more unmethylated CpG oligodinucleotides, granulocyte-macrophage colony-stimulating factor (GM-CSF), interleukin 12 (Il-12), agonists of toll-like receptors 9 (TLR9), or any other suitable adjuvant or any combination of two or more thereof. However, in some embodiments, one or more adjuvants may be provided in a separate composition that an rAAV particle and/or nucleic acid composition described herein. In some embodiments, an adjuvant composition may be administered along with (e.g., simultaneously or concurrently with) an rAAV particle and/or nucleic acid composition described herein.

In some embodiments, a composition also comprises one or more chemotherapeutic or other anti-cancer agents (e.g., cytotoxic compounds, therapeutic antibodies, or other agents). However, in some embodiments, one or more anti-cancer agents may be provided in a separate composition that an rAAV particle and/or nucleic acid composition described herein. In some embodiments, an anti-cancer agent may be administered along with (e.g., simultaneously or concurrently with) an rAAV particle and/or nucleic acid composition described herein.

### Methods

Aspects of the disclosure relate to methods of delivering a nucleic acid to a (e.g., in an rAAV particle described herein) to a subject in order to in an immune response, for example a protective immune response. In some embodiments, a composition described herein is administered to a subject at risk for cancer or having cancer (e.g., a subject diagnosed with cancer).

In some embodiments, the method comprises administering a rAAV particle as described herein or a composition as described herein to a subject via a suitable route to promote an immune response.

In some embodiments, a subject is a mammal. In some embodiments, a subject is a human subject. In some embodiments, a subject is a companion animal (e.g., a dog, a cat, or other companion animal). In some embodiments, a subject is a farm animal (e.g., a horse, cow, sheep, or other farm animal). However, aspects of the disclosure can be used to treat other animals (e.g., other mammals).

Accordingly, aspects of the disclosure relate to methods of treating cancer. In some embodiments, the method comprises administering a therapeutically effective amount of an rAAV particle or a composition as described herein to a subject having or diagnosed wcancer.

Non-limiting examples of cancer that can be treated according to methods described herein include lymphoma, hemangiosarcoma, mast cell tumors, osteosarcoma, melanoma, prostate cancer, thyroid cancer, liver cancer, kidney cancer, ocular cancer, head or neck cancer, lung cancer, gastrointestinal cancers, urogenital cancers, or cancers of other tissues or organs.

To “treat” a disease as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. The desirable result will depend upon the active agent being administered. For example, an effective amount of rAAV particles may be an amount of the particles that are capable of transferring an expression construct to a host organ, tissue, or cell (e.g., dendritic cells, macrophages, progenitor cells, for example a CD14+ monocytes, or CD34+ hematopoietic cells, or combinations thereof).

A therapeutically acceptable amount may be an amount that is capable of treating a disease, e.g., a cancer, by stimulating an immune response that can help treat the disease (e.g., alone or in combination with one or more additional anti-cancer therapies such as chemotherapy, monoclonal antibody treatment, hormonal treatment, radiation, surgery or other treatment).

As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

The rAAV particle or nucleic acid vector may be delivered in the form of a composition, such as a composition comprising the active ingredient, such as a rAAV particle described herein, and a pharmaceutically acceptable carrier as described herein. The rAAV particles or nucleic acid vectors may be prepared in a variety of compositions, and may also be formulated in appropriate pharmaceutical vehicles for administration to human or animal subjects.

In some embodiments, the number of rAAV particles administered to a subject may be provided in a composition having a concentration on the order ranging from 10⁶ to 10¹⁴ particles/mL or 10³ to 10¹⁵ particles/ml, or any values therebetween for either range, such as for example, about 10⁶, 10⁷, 10⁸, 10⁹, 10¹⁰, 10¹¹, 10¹², 10¹³, or 10¹⁴ particles/ml. In one embodiment, rAAV particles of higher than 10¹³ particles/ml are administered. In some embodiments, the number of rAAV particles administered to a subject may be on the order ranging from 10 to 10 vector genomes(vgs)/ml or 10 to 10 vgs/ml, or any values there between for either range, such as for example, about 10⁶, 10⁷, 10⁸, 10⁹, 10¹⁰, 10¹¹, 10¹², 10¹³, or 10¹⁴ vgs/ml. In one embodiment, rAAV particles of higher than 10¹³ vgs/ml are administered. The rAAV particles can be administered as a single dose, or divided into two or more administrations as may be required to achieve therapy of the particular disease or disorder being treated. In some embodiments, 0.0001 ml to 10 mls are delivered to a subject. In some embodiments, the number of rAAV particles administered to a subject may be on the order ranging from 10⁶-10¹⁴ vg/kg, or any values there between, such as for example, about 10⁶, 10⁷, 10⁸, 10⁹, 10¹⁰, 10¹¹, 10¹², 10¹³, or 10¹⁴ vgs/kg. In some embodiments, the number of rAAV particles administered to a subject may be on the order ranging from 10¹²-10¹⁴ vgs/kg.

If desired, rAAV particles may be administered in combination with other agents as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles may thus be delivered along with various other agents as required in the particular instance. Such compositions may be purified from host cells or other biological sources, or alternatively may be chemically synthesized. In some embodiments, the rAAV are delivered along with one or more adjuvants and/or one or more chemotherapeutic agents.

In certain circumstances it will be desirable to deliver rAAV particles in suitably formulated pharmaceutical compositions disclosed herein via a route that stimulates an immune response. In some embodiments, rAAV particles are delivered to the dermis or epidermis of a skin. In some embodiments, rAAV particles are delivered into a muscle of a subject. Accordingly, rAAV particles may be delivered via an intradermal, subcutaneous, and/or intramuscular injection. In some embodiments, rAAV particles are delivered to the foot pad of an animal. In some embodiments, rAAV particles are delivered by injection to one or more other tissues or organs in an amount sufficient to induce an immune response (for example a protective immune response). In some embodiments, rAAV particle are delivered orally or by inhalation (e.g., by nasal inhalation). In some embodiments, rAAV particles are delivered intraocularly, intravitreally, subretinally, parenterally, intravenously, intracerebro-ventricularly, or intrathecally.

In some embodiments, rAAV particles are not delivered systemically, for example to avoid expression in the liver or other site that could produce tolerance as opposed to an immune response.

The pharmaceutical forms of the rAAV particle compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subretinal, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions are prepared by incorporating the rAAV particles in the required amount in the appropriate solvent with several of the other ingredients enumerated above, as required, followed by filtered sterilization or another sterilization technique. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The amount of rAAV particle or nucleic acid vector compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the rAAV particle compositions, either over a relatively short, or a relatively prolonged period of time, as may be determined by the medical practitioner overseeing the administration of such compositions.

The composition may include rAAV particles, either alone, or in combination with one or more additional active ingredients, which may be obtained from natural or recombinant sources or chemically synthesized.

Toxicity and efficacy of the compositions utilized in methods of the disclosure can be determined by standard pharmaceutical procedures, using either cells in culture or experimental animals to determine the LD50 (the dose lethal to 50% of the population). The dose ratio between toxicity and efficacy is the therapeutic index and it can be expressed as the ratio LD50/ED50. Those compositions that exhibit large therapeutic indices are preferred. While those that exhibit toxic side effects may be used, care should be taken to design a delivery system that minimizes the potential damage of such side effects. The dosage of compositions as described herein lies generally within a range that includes an ED50 with little or no toxicity. The dosage may vary within this range depending upon the dosage form employed and the route of administration utilized.

### Subjects

Aspects of the disclosure relate to methods for use with a subject, such as human or non-human primate subjects. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals (e.g., companion animals) such as dogs and cats; livestock such as horses, cattle, pigs, sheep, goats, and chickens; and other animals such as mice, rats, guinea pigs, and hamsters.

In some embodiments, the subject has or is suspected of having a disease that may be treated with immunotherapy (e.g., alone or in combination with additional anti-cancer therapy). In some embodiments, the subject has or is suspected of having cancer. Subjects having cancer can be identified by a skilled medical practitioner using methods known in the art, e.g., by measuring serum concentrations of cancer-associated markers, genetic analysis, CT, PET, or Mill scans, tissue biopsies, or any combination thereof.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Efficient Transduction of Innate Immunocompetent Cells Using AAV6

Although AAV6 vectors have been used successfully in number of human cells (e.g., fibroblasts, dendritic, hematopoietic stem cells) and animal models (e.g., dog—muscle, cardiomyocytes; mouse—muscle, lungs), a number of steps in the life cycle of AAV, including intracellular trafficking and nuclear transport, continue to appear to limit the effectiveness of these vectors in gene therapy.

The substitution of specific surface exposed specific tyrosine (Y), serine (S), and threonine (T) residues on AAV6 capsids significantly increases the transduction efficiency of these vectors, through bypassing phosphorylation of the capsid by common cellular kinases, subsequent ubiquitination, and proteasome-mediated degradation (human dendritic cell, human hematopoietic stem cell, mouse glia). Phenylalanine (F) and valine (V) were used for substitution due to their opposite charge, approximate similarity of size and the lack of recognition by modifying kinases. Several capsid-optimized vectors such as AAV6-Y705-731F+T492V and AAV6-S663V were identified as the most efficient. These particular residues are localized in critical regions of the capsid: towards the base of the protrusions surrounding the icosahedral 3-fold axes of the capsid (T492V), in the depression surrounding the 2-fold axes (Y705F and Y731Y), or the HI loop (S663 V), which were shown to play important role in AAV vectors intracellular trafficking.

### Production of Recombinant AAV Vectors.

Briefly, HEK293 cells were transfected using polyethyleneimine (PEI, linear, MW 25,000, Polysciences, Inc.). Seventy-two hours post-transfection, cells were harvested and vectors were purified by iodixanol (Sigma) gradient centrifugation (multi-pump Watson & Marlow 205S) and ion exchange column chromatography (#17-1152-01 HiTrap Sp Hp 5 ml for AAV2 or #17-1154-01 HiTrap Q HP 5 mL for all other AAV, GE Healthcare) with GE Healthcare Peristaltic Pump P-1). Virus was then concentrated and buffer exchanged into Lactated Ringer's solution in three cycles using centrifugal spin concentrators (Apollo, 150-kDa cut-off, 20-ml capacity, CLP, #2015010 Orbital Bioscience or Fisher). To determine genome titers, 10 ul of purified virus were incubated with DNase I (Invitrogen) at 37° C. for 2 h, then with Proteinase K (Invitrogen) at 55° C. for an additional 2 h. The reaction mixture was purified by phenol/chloroform, followed by chloroform extraction. Packaged DNA was precipitated O/N with ethanol in the presence of 20 ul glycogen (Invitrogen). DNase I-resistant AAV2 particle titers were determined by qPCR with the following primer-pairs specific for the promoter or gene of interest (GOI) by using SYBR GreenER™ PCR Master Mix (Invitrogen).

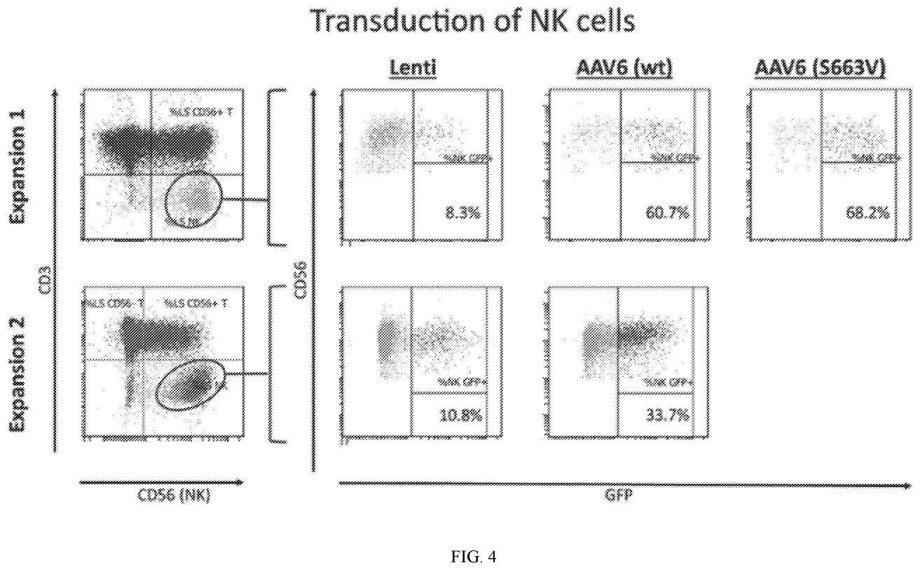

Blood is composed of myeloid and lymphoid lineage committed cells. In general, lymphoid cells are composed of B cells and T cells and natural killer cells (NK). T cells, in general, comprise adaptive cells, such as cytotoxic cells (CD8 T cells) and helper cells (CD4 T cells) and a subclass of innate cells (gamma delta (γδ) T cells). There is a need to efficiently genetically engineer lymphocyte cells, including adaptive and innate T cells and NK cells. Recent clinical data has conclusively shown that introduction of chimeric antigen sequences into adaptive T cells is an effective treatment against some cancers, specifically B-cell leukemias. The use of other cell types, particularly innate cells such as NK and γδ T cells, is much less efficient then transduction of adaptive immune cells. Lentivirus-based recombinant viruses can efficiently transduce adaptive T cells, but the transduction efficiency of innate cells is much lower. In some cases, such as discussed below for transduction of NK-92 cells, a cell line generated from primary NK cells, the transduction efficiency is less than 2% under ideal conditions. Using a serum free medium one can expand immunocompetent cells from human PBMCs, with an expansion of γδ T cells (FIG. 3, see K. Sutton et al., 2016). Transduction of γδ T cells from this human primary cell expansion with a recombinant SIN lentivirus encoding the marker protein, GFP, which is used to identify genetically modified cells, is less than 12% for two separate donors (FIG. 3). As a means to test if transduction efficiency can be increased using recombinant adeno-associated virus (AAV), recombinant AAV6 and an 5663V variant was generated. These recombinant AAV6 vectors were then used to transduce the same cultures that were inefficiently transduced with recombinant lentivirus. The efficiency of AAV6 transduction was greater than 50% for both expansions, and as high as 67% for donor 1. The AAV6-S663V vector was approximately as effective as AAV6.

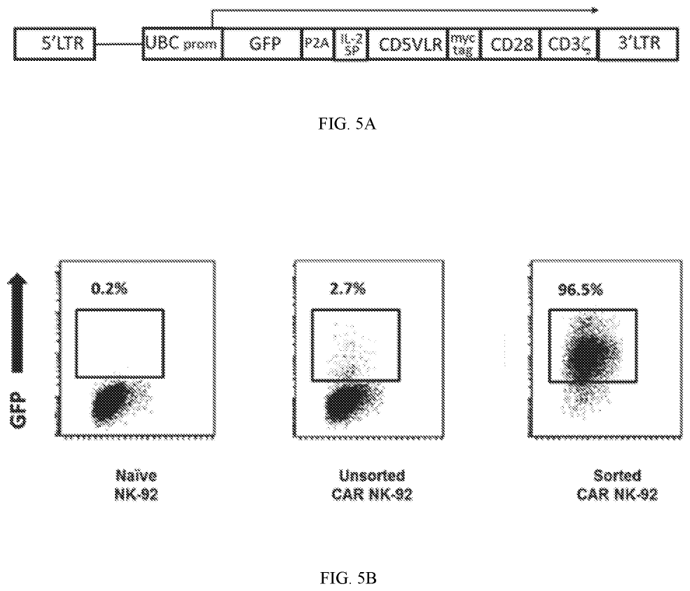

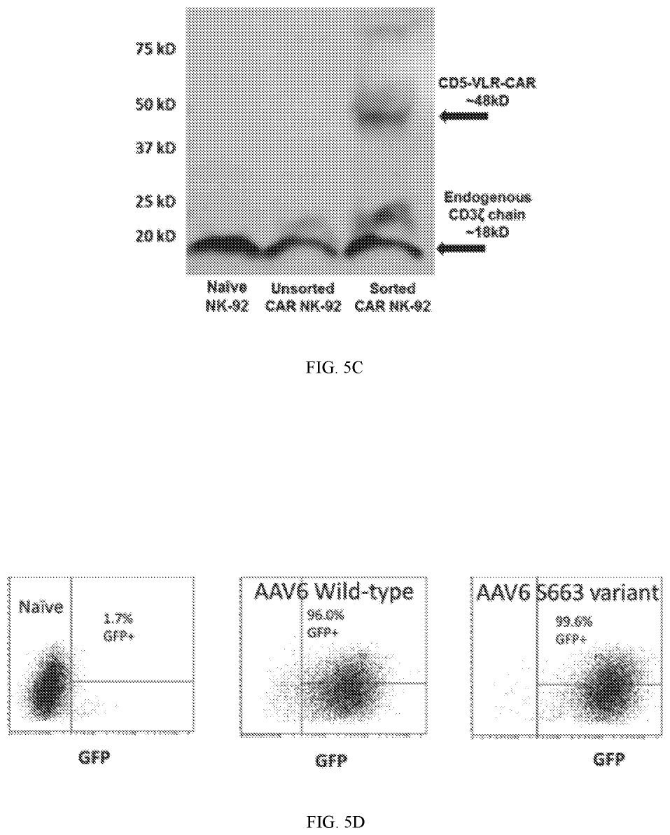

Using the same culturing system, it was possible to determine the transduction efficiency of primary human NK cells. Using a similar strategy as used for γδ T cells, NK cells can be identified in the expanded cell culture and transduction efficiency can be determined by measuring GFP expression. Compared to the transduction efficiency of lentivirus, AAV is 3-7 fold more efficient (FIG. 4). Transduction efficiency approaching 80% was achieved with the AAV-S663V variant. Together, these data show that transduction of primary innate immunocompetent cells, including γδ T cells and NK cells, are efficiently transduced and in all cases transduction efficiency is greater than that achieved with recombinant lentivirus.

In addition to transducing γδ T cells and NK cells, the transduction of an NK cell line, NK-92, that is refractory to lentivirus transduction, was tested. A high titer lentivirus was generated that encodes a GFP marker protein and an anti-CD5-CAR. The lentivirus was used to transduce NK-92 cells and transduction efficiency and transgene expression was monitored by flow cytometry and western blotting. Transduction efficiency was extremely low, less than 3% using lentivirus, and there was no evidence of transgene expression. GFP positive cells were then cell sorted to approximately 90% and the cells were expanded. Only when these cells were sorted and expanded could transgene expression be observed. In contrast, AAV6 and two variants were used to transduce the same cell line, and transduction efficiencies were greater than 90%.

Overall, it is shown that AAV6 can be used to effectively and efficiently transduce innate immunocompetent cells, which includes NK and γδ T cells. Transduction efficiency of primary innate cells is substantially higher than transduction efficiencies with a lentiviral vector. Therefore, AAV6 provides a major advantage to engineering these important subsets of immunocompetent lymphoid cells.

### Mutations of Surface-Exposed Residues on the AAV6 Capsid

In certain embodiments, this disclosure relates to a non-naturally nucleic acid encoding an AAV6 capsid protein sequence as provided in SEQ ID NO: 1 with one or more of the following mutations: T251V, D328I, S455V, G470V, S472V, V489I, T492V, D495V, D495I, S499V, H527T, W503I, D530V, K531E, V540I, S551V, D590V, K666E, K567E, I648V, S663V, A664V, T665V, S669V, E689D, Y705F, A706I, A709V, A709I, V711I, V715I, T722V, Y731F, or combinations thereof. The first letter matches to the amino acid in capsid of WT-AAV6 and the number corresponds to amino acid position on VP3 that was mutated, and the last letter is the substituting amino acid: alanine (A), aspartate (D), tyrosine (Y), serine (S), threonine (T), lysine (K), glutamine (E), isoleucine (I), tryptophan (W), histidine (H), glutamine (G). These mutation change in several important structural components of AAV capsids and impart certain overall capsid properties such as localized towards the base of the protrusions surrounding the icosahedral 3-fold axes of the capsid (T492S, S551V and K531E), in the depression surrounding the 2-fold axes (Y705F and Y731Y)—both important for AAV packaging, or the HI loop (Y663S, A664V, T665V)—escape from endosome; minimizing capsid phosphorylation and possible proteosomal degradation (all Y-to-F, T-to V, S-to-V, K-to-E and E689D); increase hydrophobicity of the capsid (all substitutions with V or I)); change charge (any H, K or D substitutions), reduce binding affinity to HSPG (K531E); possible minimizing antibody neutralization (H527T, W503I, A70V).

Some mutations showed favorable phenotype for other AAV serotypes and in some other cell types. Mutation can be used as single as well as in combination of 2, 3, 4 etc. mutations together on same capsid.

### γδ T Cells in Immunotherapy:

γδ T cells are one of the three immune cell types that express antigen receptors. They contribute to lymphoid antitumor surveillance and bridge the gap between innate and adaptive immunity. γδ T cells have the capacity of secreting abundant cytokines and exerting potent cytotoxicity against a wide range of cancer cells. γδ T cells exhibit important roles in immune-surveillance and immune defense against tumors and have become attractive effector cells for cancer immunotherapy. γδ T cells mediate anti-tumor therapy mainly by secreting pro-apoptotic molecules and inflammatory cytokines, or through a TCR-dependent pathway. Recently, γδ T cells are making their way into clinical trials. Some clinical trials demonstrated that γδ T cell-based immunotherapy is well tolerated and efficient.

The use of a variant of an adeno-associated virus (S663V AAV-6) incorporating MND promoter resulted in transduction efficiencies of over 70% percent for NK and γδ T cells. This is a significant improvement to previous method using lentiviral vectors in serum free media, which transduced γδ T cells with an efficiency of approximately 20%. (Cytotherapy. 18(7): 881-892, 2016). Recombinant AAV6 (rAAV6) with the substitution of surface serine, tyrosine, and threonine residues has shown increased transduction efficiencies. The primary application of this technology is expression of engineered CAR in NK and γδ T cells.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of delivering a recombinant nucleic acid to innate immune cells, the method comprising contacting natural killer cells with a recombinant adeno-associated virus 6 (rAAV6) comprising a recombinant nucleic acid encoding a VP3 capsid protein.

2. The method of claim 1, wherein the rAAV6 VP3 capsid protein comprises a non-serine amino acid residue at a position corresponding to S663 of the wild-type AAV6 capsid protein as set forth in SEQ ID NO:1.

3. The method of claim 2, wherein the non-serine amino acid residue at a position corresponding to S663 of the wild-type AAV6 capsid protein as set forth in SEQ ID NO:1 is valine.

4. The method of claim 1, wherein the nucleic acid is an expression construct that is flanked on each side by an inverted terminal repeat sequence.

5. The method of claim 1, wherein the recombinant nucleic acid encodes a chimeric antigen receptor (CAR).

6. The method of claim 1, wherein the nucleic acid is a single-stranded or self-complementary rAAV nucleic acid vector.

7. The method of claim 1, wherein the recombinant nucleic acid is delivered via direct injection of rAAV6.

8. A method of delivering a recombinant nucleic acid to innate immune cells, the method comprising contacting gamma delta T cells with a recombinant adeno-associated virus 6 (rAAV6) comprising a recombinant nucleic acid encoding a VP3 capsid protein.

9. The method of claim 8, wherein the rAAV6 VP3 capsid protein comprises a non-serine amino acid residue at a position corresponding to S663 of the wild-type AAV6 capsid protein as set forth in SEQ ID NO:1.

10. The method of claim 9, wherein the non-serine amino acid residue at a position corresponding to S663 of the wild-type AAV6 capsid protein as set forth in SEQ ID NO:1 is valine.

11. The method of claim 9, wherein the nucleic acid is an expression construct that is flanked on each side by an inverted terminal repeat sequence.

12. The method of claim 9, wherein the recombinant nucleic acid encodes a chimeric antigen receptor (CAR).

13. The method of claim 9, wherein the nucleic acid is a single-stranded or self-complementary rAAV nucleic acid vector.

14. The method of claim 9, wherein the recombinant nucleic acid is delivered via direct injection of rAAV6.

15. A composition comprising natural killer cells, wherein the natural killer cells comprise a recombinant AAV genome that encodes a chimeric antigen receptor operably connected to a promoter that is active in natural killer cells.

16. A method of treating cancer comprising administering an effective amount of a compositions of claim 15 to a subject in need thereof.

17. The method of claim 16, wherein the composition is administered in combination with a chemotherapy agent.

18. A composition comprising gamma delta T cells, wherein the gamma delta T cells comprise a recombinant AAV genome that encodes a chimeric antigen receptor operably connected to a promoter that is active in gamma delta T cells.

19. A method of treating cancer comprising administering an effective amount of a compositions of claim 18 to a subject in need thereof.

20. The method of claim 19, wherein the composition is administered in combination with a chemotherapy agent.


SEQUENCE LISTING (1 sequence)

SEQ ID NO: 1 - protein, 736 aa, Artificial
  Synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYFLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL